SEQUENCE LISTING (8 sequences)

SEQ ID NO: 1 - protein, 15 aa, Unknown
  Fungal
APAAEVESVAVEKRQ

SEQ ID NO: 2 - protein, 7 aa, Unknown
  Fungal
VPLEERQ

SEQ ID NO: 3 - protein, 17 aa, Unknown
  Fungal
SCRPAAEVESVAVEKRQ

SEQ ID NO: 4 - protein, 19 aa, Unknown
  Fungal
MYRKLAVISAFLATARAQS

SEQ ID NO: 5 - protein, 18 aa, Unknown
  Fungal
MVSFTSLLAASPPSRASC

SEQ ID NO: 6 - protein, 21 aa, Unknown
  Fungal
MVSFTSLLAGVAAISGVLAAP

SEQ ID NO: 7 - protein, 20 aa, Unknown
  Fungal
MIVGILTTLATLATLAASVP

SEQ ID NO: 8 - protein, 21 aa, Unknown
  Fungal
MNKSVAPLLLAASILYGGAAA

(19) United States
(12) Patent Application Publication
(10) Pub. No.: US 2011/0053218 A1
(43) Pub. Date: Mar. 3, 2011

(54) MULTIPLE PROMOTER PLATFORM FOR PROTEIN PRODUCTION

(75) Inventors: Valentino Setoa Junior Te'O, New South Wales (AU); Kaisu Milja Helena Nevalainen, New South Wales (AU)

(73) Assignee: APPLIMEX SYSTEMS PTY LTD., Macquarie University ,NSW (AU)

(21) Appl. No.: 12/809,396

(22) PCT Filed: Dec. 16, 2008
(86) PCT No.: PCT/AU2008/001847

§ 371 Date: Oct. 26, 2010

(30) Foreign Application Priority Data
Dec. 19, 2007 (AU) 2007906984

(51) Int. Cl. C12P 21/00; C12N 15/80; C12N 1/15

(52) U.S. Cl. 435/69.1; 435/320.1; 435/254.11; 435/254.6

(57) ABSTRACT

A multiple promoter platform for expression of a protein or multiple and different proteins in a microorganism comprising two or more expression vectors having a nucleic acid molecule encoding a protein to be expressed, or having different nucleic acid molecules encoding different proteins to be expressed, wherein each vector has a different promoter operably linked to the nucleic acid molecule, and uses of the multiple promoter platform to produce recombinant proteins.

### TECHNICAL FIELD

The present invention relates to an expression system driven by a selection of different promoters, a process for high level expression and secretion of proteins from a host, DNA recombinant vectors, and transformed host strains.

### BACKGROUND

Production of gene products in microbial cells, mammalian cell lines and plant expression systems have traditionally been carried out using an expression vector where the gene of interest is expressed under the control of a single-type of promoter. Consequently, the transformed host cells carry either a single copy or multiple copies of the introduced vector for the expression of the recombinant product of interest, either stably integrated into the host genome or present as an autonomous self-replicating plasmid. Protein yields vary depending on the gene promoter, gene to be expressed, gene copy number, cultivation conditions and the expression host.

Some examples of gene expression work in different expression hosts, but using a single-type of promoter, include high level production of fungal proteins such as phytase from Aspergillus niger (˜2 g/l) and a xylanase 2 from Humicola grisea var. thermoidea (˜0.5 g/l), in the high protein-secreting filamentous fungus, Trichoderma reesei. Other examples include the production of human serum albumin (HSA) in yeasts such as Saccharomyces cerevisiae (150 mg/l) and Pichia pastoris (˜10 g/l). Examples in baculovirus-insect expression systems include work on Trichoplusia and Spodoptera frugiperda where expression of mammalian proteins such as human collagenase IV and human proapolipoprotein Al, reached levels of 300 mg/l and 80 mg/l, respectively. Furthermore, some examples using mammalian systems have resulted in production levels of 110 mg/l for humanized anti-1 BB monoclonal antibodies in Chinese hamster ovarian cells, and 1 mg/l for human laminin protein in human embryo kidney cells, respectively.

In light of the above examples and in an overall comparison with other available recombinant expression systems for proteins of interest, filamentous fungi are considered to perform well and provide a potentially a high yielding and relatively cheap option sought after in the industry.

A major and widely adopted approach for enhancing recombinant gene expression in filamentous fungi and other organisms employs the use of a single type strong promoter. Among the strongest known inducible promoters, regulated by carbon catabolite repression, are the glucoamylase A promoter (glaA) of A. niger var. awamori and the T. reesei cellobiohydrolase 1 (cbh1) promoter. A constitutive promoter used across various fungal species is the A. nidulans glyceraldehyde-3-phosphate dehydrogenase promoter (gpdA). To date, natural constitutive or carbon catabolite repression insensitive promoters comparable in strength to the cbh1 and glaA have not been described.

Typically, over-expression and secretion levels of a gene product into the growth medium of a host microorganism can be further influenced by the copy-number and genomic integration site of the expression cassette(s). Other approaches to improve expression include changing of codon usage of the incoming gene to suit that of the expression host and fusing the protein of interest to a dominant homologous secreted protein which can also help in mRNA transcription stability and utilise host expression of chaperones for correct protein folding. However, regardless of the progress made in these different areas, the expression level of the target gene product is still dictated by the use of one type of promoter and its regulatory factors that may limit the expression regardless of the copy number. Recent studies of the minimal effect from multi-copy-strains on expression, of gene products whilst under the control of a single-type of promoter, where multicopy transformants co-expressing two proteins under the control of the Kluyveromyces lactis lactase promoter (PLAC4-PBI) resulted in low yields, 75 mg/l, for secreted human serum albumin (HSA) and 65 mg/l for an Escherichia coli maltose binding protein (MBP), respectively. A two-fold improvement in endoglucanase I production in T. reesei was demonstrated between transformants with one and three copies of the endogenous endoglucanase 1 gene expressed under the cbh1 promoter. However, there was no obvious difference in expression levels between transformants harbouring two or three copies of the gene.

Gordon et al, 2001, FEMS Micronbiology Letters 194: 229-234, demonstrated the expression of glucoamylase (glaA) from A. niger under the control of its own promoter as well as under control of the trypsin-like protease promoter from Fusarium oxyporum. However, protein expression levels were regulated by changing the pH to control the promoters during expression of GlaA. Furthermore, no increase in production of the Glucoamylase protein was demonstrated in the double transformant at two pHs of 4.5 (optimal for glaA promoter) and 6.0 (optimal for the trypsin-like promoter), when compared to the transformant with glaA expressed under the single, trypsin-like protease promoter. Gordon et al screened a further 16 double transformants and reported that the single promoter transformant actually produced more GAM.

Moralejo et al, 1999, Applied Environmental Microbiology 65:1168-1174 demonstrated the use of two different promoters (gdhA, glutamate dehydrogenase, and cesB, B2 wide-spectrum esterase). Initial production of thaumatin when both promoters were present was similar to that observed under the cesB promoter alone. It was found that two promoters did have any enhanced or unexpected affect in protein production.

As a whole, the examples described above clearly highlight the limitations in the amount of protein produced in experiments using a single-type of promoter, notwithstanding the copy number or the integration site of the expression DNA cassette.

The industrially-exploited filamentous fungus T. reesei has a long history in industrial enzyme production. High levels of total proteins secreted from T. reesei have been reported to be in excess of 100 g/l. The majority of the native secreted proteins in T. reesei typically comprise the main Cellobiohydrolase I (CBHI), Cellobiohydrolase II (CBHII), Endoglucanases I, II, III IV and V (EGLI, EGLII, EGLIII, EGLIV and EGLV), and Xylanases I, II, Ill and IV (XYNI, XYNII, XYNIII and XYNIV). Accordingly, genes encoding these enzymes can provide strong promoters for the gene expression of other genes. While the above promoters are induced in the presence of cellulose and/or hemicellulose and share a number of regulatory factors, a selection of promoters under differential regulation can also be isolated. An example of a promoter regulated differently to the promoters listed above is the hex1 gene promoter from T. reesei. The expression of hex1 on cellulase-inducing medium peaked strongly within 24 hrs of growth, but the protein was still being expressed at a lower and more consistent level in a medium containing glucose that is repressor for cellulase production. Therefore, the hex1 promoter can be applied either together with cellulose-inducible promoters or as part of promoter combination used for gene expression on glucose.

The present inventors have now developed improved expression systems.

### SUMMARY OF INVENTION

In a first aspect, the present invention provides a multiple promoter platform (MPP) for expression of a protein or gene product in a microorganism comprising two or more expression vectors, each vector having a nucleic acid molecule encoding the protein or gene product to be expressed, wherein each vector has a different promoter operably, linked to the nucleic acid molecule.

The multiple promoter platform can employ two, or three, or four, or five or six vectors. Preferably, the multiple promoter platform employs three vectors.

Preferably, two or more expression vectors are selected from pHEX1, pCBH1corlin, pCBH2sigpro, pCBH2cbmlin, pXYN2sigpro, pEG2sigpro, pEG2cbmlin, or pHEN54RQ as herein defined.

Preferably the expression vectors comprise promoter regions in operative association with one or more of secretion signals, pro- and/or core-regions of endogenous proteins, or selection markers.

Preferably, the expression vectors are circular plasmids or linear DNA expression cassettes.

In a second aspect, the present invention provides a method of producing a protein comprising:

introducing into a cell a multiple promoter platform (MPP) according to the first aspect of the present invention to form a host cell; and

culturing of the host cell such that the protein of interest is expressed.

The combination of two or more expression vectors allows greater production of the protein of interest compared with host cells containing individual vectors.

Examples of conditions for suitable promoter induction include, but are not limited to, induction by the supply of nutrients such as energy or carbon source or environmental conditions such as heat, pH, starvation, and the like.

The expression of the protein is preferably induced in the host cell. Preferably, induction is by culture medium containing desired substrates. Examples include, but not limited to, cellulosic substrates such as crystalline and amorphous cellulose, cellulose oligosaccharides such as sophorose and cellobiose, hemicellulose such as xylan, glucuronoxylan, arabinoxylan, glucomannan, xyloglucan and lactose, and the protein-based substrate, soy-bean flour.

Preferably, the method further comprises obtaining or purifying the protein from the culture medium.

Preferably, the host cell is transformed with two or more different expression vectors, each having a different promoter. The vectors can be based on bacterial, fungal or other nucleic acid sources such as plasmids, chromosomal DNA or synthetic sequences.

Preferably, the host cell is transformed with two, three, four, five, or six or more expression vectors, each having a different promoter.

Preferably, the host cell is a filamentous fungal, yeast, mammalian, plant or bacterial cell. More preferably, the host cell is a filamentous fungal cell. Still more preferably, the host cell is a filamentous fungal species selected from the group consisting of Ophiostoma sp, Trichoderma sp, Aspergillus sp., Penicillium sp., Topylocladium sp., Fusarium sp., Chrysosporium sp., Magnaporthe sp., Neurospora sp., Claviceps sp., Mycosphaerella sp., Collectotrichum sp. Ustilago sp., Podospora sp. and Mucor sp.

Preferably, the filamentous fungus is Trichoderma reesei. Still more preferably, the filamentous fungus is Trichoderma reesei Rut-C30 (ATCC#56765).

Preferably, the two or more expression vectors (as circular plasmids or as linear DNA expression cassettes) are introduced into the host cell by transformation. Examples of suitable introduction methods include electroporation, protoplast transformation, biolistic bombardment, Agrobacterium-mediated transformation, DNA injection, Ti-plasmid-mediated transformation, calcium phosphate DEAE-dextran and liposome-mediated transformation, microinjection, and application of alkaline cations (e.g. LiCl).

Preferably, protoplast transformation, biolistic bombardment and electroporation are used for transformation of Trichoderma reesei Rut-C30 (ATCC#56765).

Preferably, the promoter regions are derived or obtained from the promoters selected from hex1, cbh1, cbh2, xyn2, xyn1, xyn3, egl1, egl2, egl3, egl4, egl5 and apr1.

More preferably, the promoter regions are from hex1, cbh1, cbh2, egl1, egl2, xyn1 or xyn2.

Preferably, the secretion signal has a sequence selected from CBH1, CBH2, XYN1, XYN2, EGL1, EGL2, EGL3, EGL4, and EGL5.

More preferably, the secretion signal is from CBH1, CBH2, XYN1, XYN2, EGL1 or EGL2.

Preferably, the selection marker is selected from amdS, phleo, hphB, als, agrB, trp1 and pyr4.

Preferably the pro-region is derived from CBH2, XYN1 or XYN2. More preferably, the pro-regions have the following sequences, APAAEVESVAVEKRQ (SEQ ID NO 1), VPLEERQ (SEQ ID NO: 2), SCRPAAEVESVAVEKRQ (SEQ ID NO: 3).

Preferably, the protein of interest is secreted from the transformed cells.

Preferably, the secreted protein is selected from the group consisting of enzymes, cell signalling and ligand binding proteins, peptide hormones, structural proteins, proteins or peptides of therapeutic/pharmaceutical importance such as antibodies, human growth factor, tissue plasminogen activator or any other polypeptide having potential commercial use.

Preferably, the secreted protein is selected from hydrolytic enzymes such as phytases, cellulases, xylanases, beta-glucanases, amylases, lipases, mannanases, galactosidases, arabinosidases, glucuronidases, acetyl esterases, xylosidases, mannosidases, glucosidases and proteases; antibodies, human growth factor and tissue plasminogen activator, or any other polypeptide having potential commercial use.

In a third aspect, the present invention provides a protein produced by the method according to the second aspect of the present invention.

In a fourth aspect, the present invention provides a host cell comprising two or more expression vectors, each vector comprising a nucleic acid molecule encoding the same protein, wherein the vectors have a different promoter operably linked to the nucleic acid molecule, and expression of the protein in the host is greater than the amount of the protein expressed by each vector individually in a similar host.

In a fifth aspect, the present invention provides a host cell comprising two or more expression vectors, each vector comprising a different nucleic acid molecule encoding different proteins, wherein the vectors each have a different promoter operably linked to the nucleic acid molecule, and expression of two or more protein can be performed in the same host or multiple host strains.

In a sixth aspect, the present invention provides an expression vector kit containing two or more expression vectors forming a multiple promoter platform (MPP) according to the first aspect of the present invention.

In a seventh aspect, the present invention provides a host cell transformed with two or more expression vectors forming a multiple promoter platform (MPP) according to the first aspect of the present invention.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this specification.

In order that the present invention may be more clearly understood, preferred embodiments will be described with reference to the following drawings and examples.

### BRIEF DESCRIPTION OF THE DRAWINGS

FIG. 1. 2D-PAGE analysis (using pI strip of 3-6) of secreted proteins from a T. reesei Rut-C30-XynB transformant cultivated on a minimal medium (Penttilä et al., 1987, Gene, 61:155-164) supplemented with Avicel cellulose, soy-bean flour hydrolysate and lactose. Also highlighted are the selected proteins with their identification following MALDI-TOF analysis.

FIG. 2. Schematic presentation of pCBH1corlin, pCHBIIcmblin, pCHBIIsigpro, pEGLIIcbmlin, pEGLIIsigpro, pXYNIIsigpro, pHEX1 and pHEN54RQHex1 DNA constructions used for gene expression. The position of the truncated terminator is shown as tt, while the carbohydrate binding module is labelled as cbm. The gene(s) to be expressed can be inserted at the multiple cloning sites.

FIG. 3. SDS-PAGE analysis of secreted proteins from small-scale cultures of T. reesei non-transformant and the different T. reesei-XynB transformants, following a heat-precipitation step at 70° C. for 3 hr, to remove background T. reesei mesophilic proteins. M, shows the protein markers. Lane 1, Rut-C30 (non-transformant), lanes 2-6 represent samples from transformants H1.3, RtB11, XS-4, H1.3-D3 and H1.3-D3-B4, respectively. Multiple forms of XynB are indicated by arrows.

FIG. 4. Zymogram in-gel activity analysis of T. reesei non-transformant and the different T. reesei-XynB transformants. M, shows the protein markers. Lanes 1-6 represent the non-transformant Rut-C30, and transformants H1.3, Rt-B11, XS-4, H1.3-D3 and H1.3-D3-B4, respectively.

FIG. 5. Relative fluorescence measurements from culture supernatants of different T. reesei strains with venus expressed under a single and multiple gene promoters. Mpp3, is the transformant with venus expressed under the egl2 only gene promoter, MpC4, is a transformant with venus expressed under the cbh2 only gene promoter, MpX4, is a transformant with venus expressed under the xyn2 only gene promoter and Mpp3.3 and Mpp3.5 are transformants with venus expressed under three gene promoters (egl2, cbh1 and xyn2). The background fluorescence from the medium of the non-transformant control (RutC-30) was first subtracted from fluorescence values obtained from the different T. reesei-venus transformants. Error bars are equivalent to 2 standard deviations (3 replicates).

### Mode(s) for Carrying Out the Invention

To demonstrate the present invention, a host system using T. reesei was developed. A high protein-secreting T. reesei host was transformed with a suite of two or more expression cassettes and/or vectors containing a nucleic acid molecule encoding a protein of interest, each vector with the nucleic acid to be expressed being under the control of a strong but different promoter derived from a gene encoding a dominantly-expressed endogenous protein of the host. It has been found that a greater than additive increase of the yield of the expressed protein was obtained. An added feature of this invention is simplicity, whereby the nucleic acid encoding the target proteins were all expressed in abundance by the expression host under the same cultivation condition. Therefore, this approach capitalises on the whole breadth of the promoters available for gene expression using a particular cultivation condition such as growth on cellulosic or hemicellulosic substrates. The typical and well established high level production and secretion of the various forms of non-recombinant cellobiohydrolases, endoglucanases and xylanases under such culture conditions suggests that the T. reesei gene expression machinery and hyphae can cope with such high level protein production.

In one embodiment, the present invention provides a method of producing a protein of interest. The method generally comprises transforming a host cell with two or more expression vectors comprising a promoter region in operative association with a secretion sequence, a core or pro-region of an endogenous or exogenous secreted protein, a selection marker and a gene encoding a protein of interest, wherein the expression vectors comprise different promoter regions. The host cells are cultured in a medium under conditions that will induce expression of the selected suite of gene promoters under the same culture conditions which the protein of interest is expressed and the protein of interest is obtained from the culture medium.

The present inventors have found the host Trichoderma to be particularly suitable for the present invention. In order to demonstrate the present invention, the host T. reesei Rut-C30 (ATCC# 56765) was used.

Examples of promoters include, but are not limited to hex1, cbh1, cbh2, xyn1, xyn2, xyn3, xyn4, egl1, egl2, egl3, egl4, egl5 and apr1.

Examples of secretion signals include, but are not limited to, CBHI, CBHII, XYNI, XYNII, XYNIII, XYNIV, EGI, EGII, EGIII, EGIV and EGV secretion signals.

Preferably the secretion signal has the sequence MYRKLAVISAFLATARAQS from CBHI (SEQ ID NO: 4), MVSFTSLLAASPPSRASC from XYNI (SEQ ID NO: 5), MVSFTSLLAGVAAISGVLAAP from XYNII (SEQ ID NO: 6), MIVGILTTLATLATLAASVP from CBHII (SEQ ID NO: 7), and MNKSVAPLLLAASILYGGAAA from EGII (SEQ ID NO: 8).

A variety of proteins of interest may be produced in increased amounts using the method of the present invention. Examples of such proteins include, but are not limited to industrially relevant enzymes, cell signalling and ligand binding proteins, and antibodies.

Choices of selection markers are well known to those skilled in the art and include genes (synthetic or natural) that provide the transformed cells with the ability to utilise a metabolite that is not normally metabolised by the host cell or an antibiotic resistance. Also, if the host cell lacks a functional gene for the marker chosen, then that gene may be used as a marker. Examples of selection markers include, but are not limited to amdS, ble, hphB, als, agrB, trp1, and pyr4.

### Methods

### Bacterial and Fungal Strains

The Escherichia coli strain DH5α was used as host for plasmid amplification and was propagated on L-agar plates containing Ampicillin at 100 μg/ml.

The filamentous fungus Trichoderma reesei strain Rut-C30 (ATCC# 56765), was used as an expression host for the expression of the different gene products described in this specification, and was propagated on potato dextrose agar (PDA) plates.

Identification of Strong Promoters by 2-D Gel Analysis of the Proteins from T. Reesei

Following a 7 day cultivation on the medium described under fungal cultivation, supernatant from a T. reesei-XynB transformant was analysed using 2-D gel electrophoresis as described in Grinyer et al., 2005, Curr. Genet. 47:381-388. Selected proteins were excised from the 2-D gel and subjected to tryptic digestion before performing mass assisted laser desorption ionization-time of flight (MALDI-TOF) analysis at the Australian Proteome Analysis Facility (APAF) located at Macquarie University, NSW 2109, Australia. For protein identification, peptide masses were searched using the SWISS-PROT database with ProteinLynX, on NCBI using the MASCOT search engine (www.matrixscience) or on EXpasy (www.ExpAsy.org). Amino acid sequences from the most strongly expressed proteins were used for protein identification and the corresponding gene coding sequences were used to design primers for the isolation of the DNA encoding the corresponding target protein domains and gene promoter sequences. Isolated gene promoters and appropriate gene DNA fragments were then used in the construction of the different expression vectors as described below.

Plasmids and the Synthetic xynB and Venus Genesgene

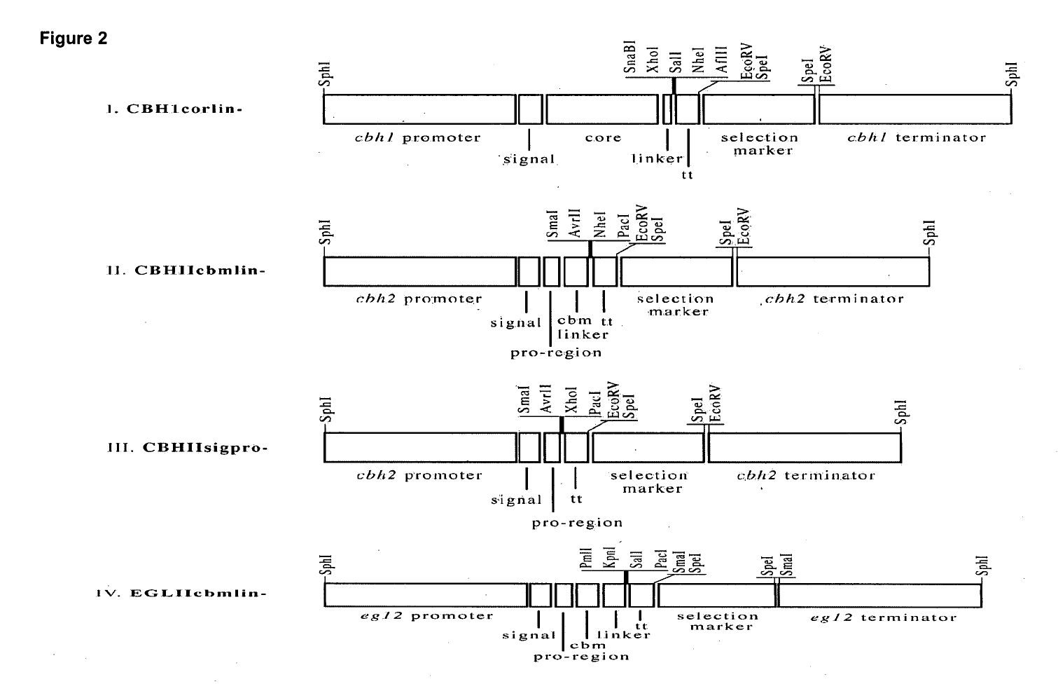

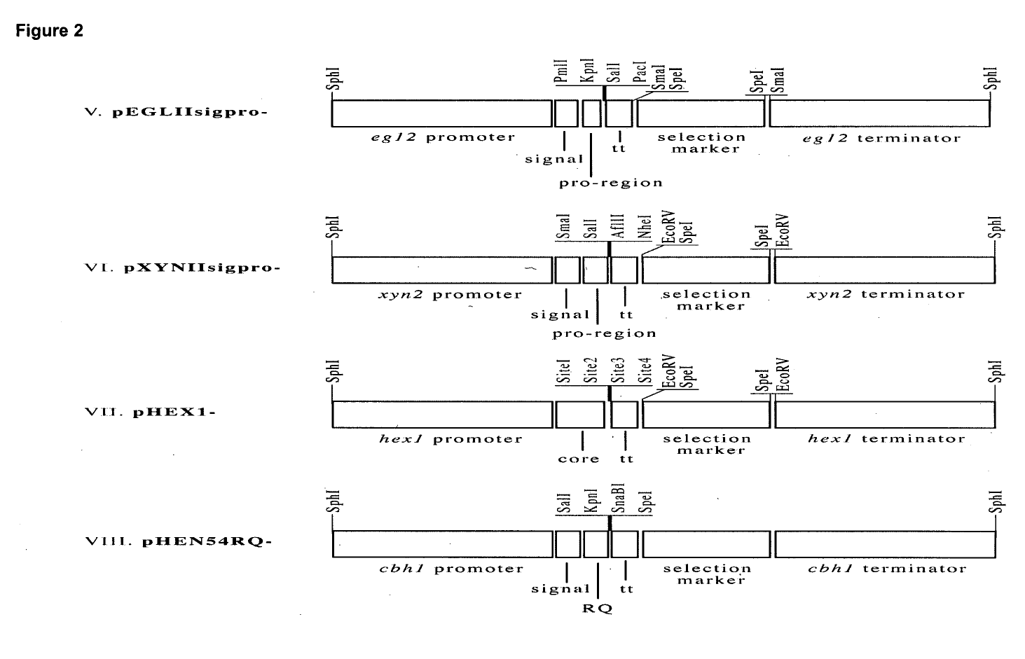

Plasmids for gene expression were designed based on the DNA sequences encoding and flanking the dominant protein HEX1 (Curach et al., 2003, Gene 331: 133-140), and the highly secreted proteins CBHI (Teen et al., 1983, Biotechnol. 1:696-699), CBHII (Teen et al., 1987, Gene, 51: 43-52), EGII (Biely et al., 1993, In Proceedings of the Trice 93 Symposium Workshop, 2-5 June, Espoo, Finland, P. Suominen and I. Reinikainen, eds, Foundation for Biotechnical and Industrial Fermentation Research, 8: 99-108) and XYNII (Törrönen et al., 1992, Bio/Technol. 10: 1461-1467). The design of the different expression DNA constructs is shown in FIG. 2. The promoter and signal sequences for each of the hex1, cbh1, cbh2, egl2 and xyn2 genes are summarized below.

DNA manipulation and cloning was carried out using the techniques outlined in Sambrook et al., 1989 (Molecular cloning: A laboratory Manual, 2nd edn. Cold Spring Laboratory Press, Cold Spring Harbor, N.Y.).

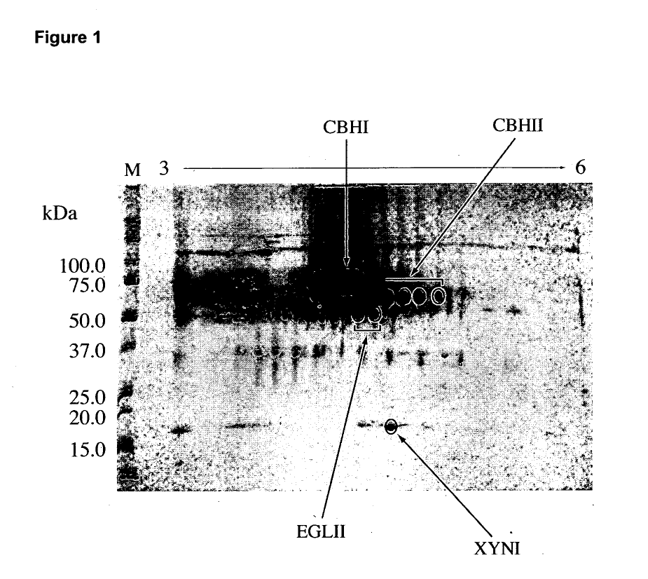

All expression vectors have 1.0-1.5 kb flanking DNA regions to facilitate homologous recombination into the fungal genome. Also included is room for insertion of selection markers (eg. amdS, ble, hphB, als, agrB, trp1, and pyr4.), whereby each expression vector can have a different selection marker. A 0.3-0.35 kb truncated terminator fragment (tt) has been inserted to ensure termination of the mRNA transcription. Furthermore, each expression vector has either a homologous pro-, core- or linker regions of a highly expressed endogenous protein (FIG. 1) incorporated into the design to facilitate proper protein folding.

The native xynB gene was initially isolated from the thermophilic bacterium Dictyoglomus thermophilum (Morris et al., 1998, Appl. Environ. Microbiol. 64: 1759-1765), and its codon usage was optimized to suit expression in Trichoderma reesei (Te'o et al., 2000, FEMS Microbiol. Lett. 190: 13-19). The synthetic xynB sequence was used in this expression work.

The venus gene (0.72 kb), encoding the Venus fluorescent protein derived from the yellow fluorescent protein, YFP, further derived from the green fluorescent protein, GFP, which was originally isolated from the eukaryote, jellyfish, Aequorea victoria (YFP, Nagai et al., 2002, Nat. Biotechnol., 20:87-90), was also used in this expression work.

### Fungal Transformation Methods

Expression vectors were introduced into T. reesei Rut-C30 using either the protoplast transformation (Penttilä et al., 1987, Gene, 61:155-164) or the biolistic bombardment delivery method (Te'o et al., 2003, J. Microbiol. Methods 51:393-399).

Single types of expression DNAs were transformed individually, into T. reesei as described above. However, in some cases, up to two or three different DNAs in equimolar ratios were also introduced into T. reesei in a single transformation step, to test for the effect of multiple promoters effect.

Following transformations and depending on the selection marker used, cells were plated out onto selective plates containing either the antibiotics Hygromycin B (60 U/ml), or Phleomycin (90 U/ml), or Sulfonyl Urea (up to 100 ug/ml), or the substrate Acetamide as a sole nitrogen source.

### Fungal Cultivation

Conidia from fungal transformants were cultivated in 50, ml minimal salt-based cultures (Penttilä et al., 1987, Gene, 61:155-164) that were supplemented with Avicel cellulose (2% w/v), Soy bean flour (1.5% w/v) and Lactose (1% w/v), pH 6.5. Following a 7 day cultivation at 28° C., supernatant was harvested by centrifugation and used for further analysis.

### Xylanase Activity Assays

The DNS-based reducing sugar assay described in Bailey and Poutanen (1989, Appl. Microbiol. Biotechnol. 30: 5-10), was used to determine xylanase B activity units in the culture supernatants. Samples were heat-treated first at 70° C. for 2.5-3 hr and centrifuged, before the assays were carried out on the resulting supernatants. Xylanase B assays were carried out at 70° C. for 5 min and activity units were expressed as BXU/ml, whereby 1 BXU is the same as 1 nkat, which is equivalent to 1 nmol of reducing sugars released by the enzyme in 1 second.

### Venus Fluorescent Protein Measurements

Following 5-7 days of incubation at 28° C., cultures were harvested by centrifugation, and 100-200 μl of clear supernatants were placed on a 96 well microtitre plate and fluorescence was measured at λ_{485/520 nm (excitation/emmission).}

Confocal Fluorescent T. reesei-Venus Images

Mycelia from selected cultures were placed on microscope slides (model J1A 7101WT, Sail, China) and images were taken using the Confocal examining microscope, Fluoview 300 (Olympus, Australia) using an Argon laser at excitation and emission wavelengths of 488 nm and 505-525 nm, respectively.

### SDS-PAGE Analysis of Secreted Proteins

Samples (10-25 μl) from culture supernatants were electrophoresed and analyzed on 4-12% gradient SDS-PAGE gels from Invitrogen (cat. no. NP0335BOX), following the company's instructions.

### Results

2-D gel analysis of secreted proteins from T. reesei

FIG. 1 shows a 2-D PAGE analysis of the culture supernatant from a T. reesei-XynB transformant grown under cellulase and xylanase-inducing conditions. Several dominantly expressed proteins were excised from the gel and subjected to MALDTI-TOF analysis for their identification as explained in the previous section. The most abundant secreted proteins were identified as CBHI, CBHII, EGII, XynI and XynII. Dominantly-expressed proteins indicate the presence of strong gene promoters driving transcription of the genes encoding them. A variety of these promoters in different combinations have been tested for the gene expression work described herein.

### Expression Vectors

Expression vectors were designed and constructed according to the gene sequences encoding and flanking the abundantly expressed and secreted proteins HEXI, CBHI, CBHII, EGII and XYNII, as identified by 2-D gel analysis. Salient features of the expression cassettes are shown in FIG. 2.

MPP Demonstration with hex1, cbh1 and xyn2 Gene Promoters

The xynB gene from the thermophilic bacterium Dictyoglomus thermophilum (Te'o et al., 2000, FEMS Microbiol Lett, 190: 13-19) was inserted into three of the eight expression vectors shown in FIG. 2 and used to transform T. reesei reesei Rut-C30. The vectors (see FIG. 2) containing the xynB-fusion DNA construct were transformed individually as circular plasmids or as linear expression DNA cassettes into T. reesei, and the resulting transformants were screened for improved XynB secretion using SDS-PAGE gel and xylanase B activity analysis procedures. Following introduction of the first expression vector, the best XynB-producing transformant was used subsequently used as a host for the introduction of the second xynB-fusion vector, and the best XynB-secreting transformant identified. This transformant was then further transformed with the third xynB-fusion cassette and the strains screened for XynB activity as described in the Methods section. Selected transformants now expressing the XynB from one, two and three different promoters were cultivated in shake flasks for comparison of the production of the thermophilic XynB under the multiple and different gene promoters. Supernatants were harvested and stored at 4° C. for further analysis. Because of the thermostable nature of XynB, samples were incubated at 70° C. for 3 hrs, to remove the mesophilic T. reesei proteins before carrying out enzyme activity assays (Table 1).

TABLE 1
Reducing sugar assay of the supernatants from the transformants
harbouring the different xynB-fusion constructions expressed
under different promoters. Xylanase activities shown here were
from samples heat-treated at 70° C. for 3 hrs before
performing the assays.

| | | Xylanase activity |
|---|---|---|
| Host strain | Promoter | (BXU/ml) |
| RutC30 | non-transformant control | 2.96 |
| H1.3 | hex1 | 10.27 |
| Rt-B11 | cbh1 | 2467.79 |
| XS-4 | xyn2 | 3052.38 |
| H1.3-D3 | hex1 and cbh1 | 2879.66 |
| H1.3-D3-B4 | hex1, cbh1 and xyn2 | 6094.93 |

Table 1 demonstrates a clear and greater than additive increase of the XynB (xylanase) activity present in the culture medium from a series of transformants containing multiple and different xynB-fusion expression cassettes. It is evident from these results that the H1.3-D3-B4 strain containing three different expression vectors featuring three different promoters (hex1, cbh1 and xyn2) had the highest xylanase activity of the transformants listed in Table 1 and that the additive effect exceeds the activity obtained by adding the activities obtained by each single type promoter. The H1.3-D3-B4 transformant has a 2.1-fold higher xylanase activity when compared to transformant H1.3-D3 which has the xynB gene expressed under two promoters only. Furthermore, the transformant H1.3-D3-B4 has 1.99-, 2.46- and 593.0-fold higher activities when compared to transformants XS-4, Rt-B11 and H1.3, respectively, which all have xynB expressed under the three individual promoters (Table 1).

Further examples that the different promoters have a direct and greater than additive effect on the expression and secretion yield of XynB are shown in Table 2. Transformant H1.3 harbouring the hex1-xynB fusion was used as the parent for transformation with the cbh1-xynB construct to give rise to transformant H1.3-D3. Later, transformant H1.3-D3 containing both the hex1-xynB and cbh1-xynB vectors was used as the parent for transformation with xyn2-xynB fusion DNA, resulting in the transformant H1.3-D3-B4. H1.3-D3-B4 produced from the same transformation lineage now contains the xynB fusions under the control of hex1, cbh1 and xyn2 gene promoters (Table 2). Therefore, the copy number and integration site (s) of hex1-xynB expression DNA is the same in transformants H1.3, H1.3-D3 and H1.3-D3-B4. Likewise the copy number and integration site (s) of the cbh1-xynB expression DNA is the same in transformants H1.3-D3 and H1.3-D3-B4. In a direct comparison, the secreted xylanase B enzyme activity from H1.3-D3-B4 has 2.1- and 593.0-fold higher activities compared to transformants H1.3-D3 containing two promoters, and to H1.3 having one promoter, respectively. Noting that since two or three vectors were used successfully; it will be appreciated that combinations of four or more vectors each with a different promoter would be suitable for the present invention.

TABLE 2
Direct comparison of transformants produced from a
single transformation lineage.

| | | Xylanase activity |
|---|---|---|
| Host strain | Promoter | (BXU/ml) |
| H1.3 | hex1 | 10.27 |
| H1.3-D3 | hex1 and cbh1 | 2879.66 |
| H1.3-D3-B4 | hex1, cbh1 and xyn2 | 6094.93 |

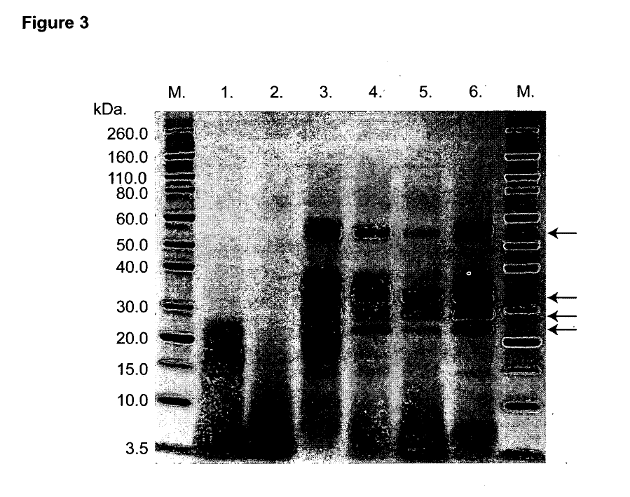

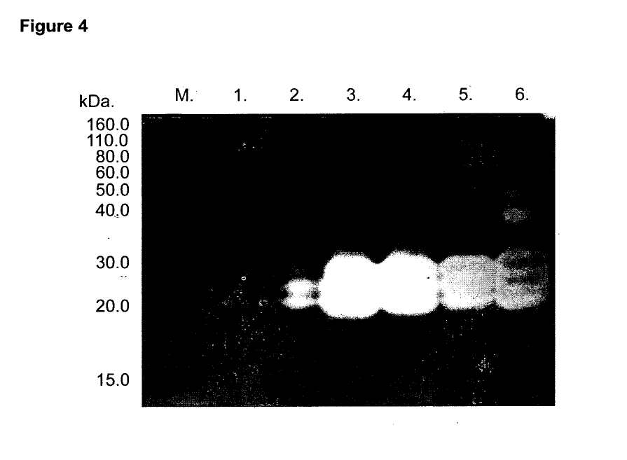

Samples of 25 μl from the culture supernatant of each strain were also were analysed on SDS-PAGE and the results are shown in FIG. 3. The expected molecular weight size of XynB is about 23 kDa. Closer examination of the SDS-PAGE gel revealed the presence of multiple proteins running between approximately at 25 to 58 kDa range in strains Rt-B11, XS-4, H1.3-D3 and H1.3-D3-B4 (highlighted by arrows, FIG. 3). XynB has up to three potential N-linked glycosylation sites and it is possible that XynB was subjected to post-translational modification (e.g. N-linked glycosylation) in the fungal host. Also, the intensity of the multiple XynB protein bands appears to be higher in strain H1.3-D3-B4 (lane 6, FIG. 3) when compared to the other strains, indicative of at least a two fold higher secreted XynB protein yield. A zymogram (in-gel) activity assay showed that even though XynB is present in most of the multiple forms, they were still highly functional, as indicated by the activity clearings around the protein bands (FIG. 4).

MPP Demonstration with xyn2, egl2 and cbh2 Gene Promoters

Similar to the xynB expression work using the hex1, cbh1 and xyn2 gene promoters discussed above, MPP was also tested but using a different combination set of gene promoters, xyn2, egl2 and cbh2.

According to the results shown in Table 3, Trichoderma transformant strains with xynB expressed under the three promoters (xyn2, egl2 and cbh2), secreted higher amounts of XynB into the cultivation medium, when compared to strains with xynB expressed under one (egl2) and two (xyn2 and cbh2) gene promoters, respectively. These results are in agreement with XynB expression results described above, on the further improvement of XynB protein production as a direct effect of having multiple promoters (two or more).

TABLE 3
Direct comparison of transformants with xynB expressed
under different combination of gene promoters.

| | | Xylanase activity |
|---|---|---|
| Host strain | Promoter | (BXU/ml) |
| EC-21 | egl2 | 2184.47 ± 240.28 |
| MPP-1 | xyn2 and egl2 | 3163.43 ± 320.37 |
| MPP-4 | xyn2, egl2 and cbh2 | 3446.60 ± 354.7 |

Demonstration of MPP-Expression with the Fluorescent Protein, Venus

To demonstrate further the effect of multiple promoters on gene expression, the gene encoding the fluorescent protein Venus derived from the yellow fluorescent protein (YFP) and ultimately of the green fluorescent protein (GFP), that was originally isolated from the eukaryote, jellyfish Aequorea victoria (Nagai et al., 2002, Nat. Biotechnol., 20:87-90), was inserted into the MPP expression vectors and tested.

T. reesei was initially transformed with the expression DNA containing venus expressed under the egl2 promoter and screened on minimal agar plates containing Acetamide as the sole nitrogen source, described in Methods. As a result, a number of T. reesei-venus transformants were produced, of which transformant labelled Mpp3 was used for further work.

Confocal fluorescent images of hyphae from the Mpp3 transformant and the non-transformant control T. reesei, RutC30, grown under inducing conditions were produced and compared. High fluorescence was detected in the Mpp3 transformant indicative of Venus expression. In contrast, only low/background fluorescence was detected in the inducing medium and hyphae from the non-transformant control, RutC30.

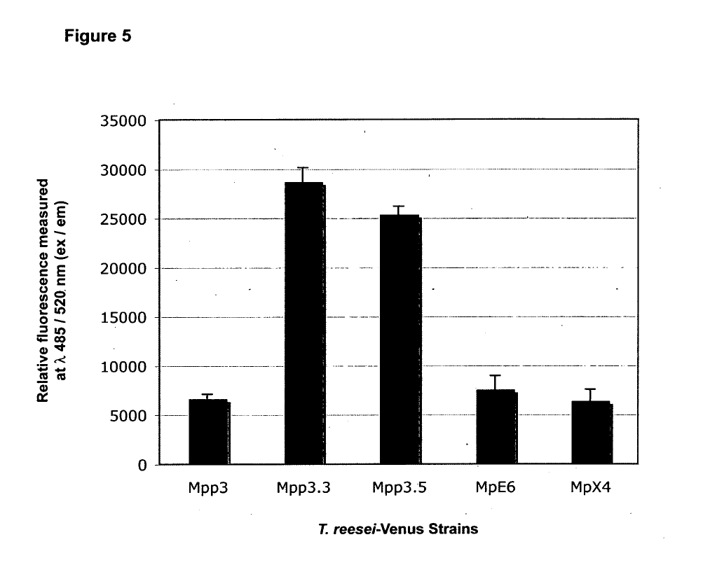

To demonstrate the effect of MPP on venus expression, the Mpp3 transformant with venus expressed under the egl2 promoter was later used as the parent strain, for further transformations. The venus gene was introduced again into the Mpp3 transformant but using expression DNAs with the venus expressed under the xyn2 and cbh1 gene promoters, and generated transformants MPP3.3 and MPP3.5 (FIG. 5).

Similar to the expression results from the bacterial and thermophilic xylanase gene (xynB) described above, the expression of the eukaryotic venus gene under the control of multiple promoters resulted in transformants producing and secreting higher Venus fluorescence (up to 4.5-fold), detected in the culture supernatants (Table 4 and FIG. 5), when compared to strains with venus under the control of a single type of promoter.

TABLE 4
Relative fluorescence of different T. reesei-Venus supernatants
measured at λ_{485/520 nm (ex/em)}.

| | | Venus Activity |
|---|---|---|
| | | (relative fluorescence |
| | | measured at 485/520 nm |
| Host strain | Promoter | (ex/em)) |
| MpX4 | xyn2 | 6401 ± 1173 |
| MpC4 | cbh2 | 6587 ± 3841 |
| Mpp3 | egl2 | 6675 ± 418 |
| Mpp3.3 | egl2, cbh1 and xyn2 | 28747 ± 1381 |
| Mpp3.5 | egl2, cbh1 and xyn2 | 25408 ± 773 |

The background fluorescence from the medium of the non-transformant control RutC30, was first subtracted from values obtained for the different T.reesei-venus transformants.

### SUMMARY

Transformants expressing a gene'product of interest from multiple and different promoters were found to produce and secrete higher amounts of protein and activities of the expressed gene products (examples, bacterial and thermophilic xylanase B, XynB, and eukaryotic fluorescent protein Venus, derived from the yellow fluorescent protein, YFP, and ultimately from the green fluorescent protein, GFP, that was originally isolated from the jellyfish, Aequorea victoria), when compared to transformants carrying a single type of expression DNA, with the gene of interest under the control of one type of promoter.

The present invention can also be applied not only to the expression of one type of gene product under different promoters, but also to different and multiple gene products, in different combinations during cultivation/growth time, in a single and/or multiple host strain(s) (eg. different filamentous fungal species or different yeast species).

The present invention can also be applied readily to other expression systems such as yeast, bacterial, mammalian cell systems and plants, where suitable promoters for high level expression are available. As demonstrated in the yeast Yarrowia lipolytica (Barth and Gaillardin, 1996, In: Nonconventional Yeasts in Biotechnology (Wolf, K., Ed, pp. 313-388. Springer-Verlag, Berlin; Nicaud et al., 2002, FEMS Yeast Research, 2:371-379), several proteins (proteases, lipases, esterases and RNAse) were secreted into the cultivation medium. Among them, the alkaline extracellular protease (AEP) was found to reach several grams per litre. As these multiple enzymes are naturally secreted by the yeast, Y. lipolytica, expression of the gene promoters controlling their expression can be used to design multiple expression vectors and subsequently used for the expression of one or multiple gene products in the same host or different species. Similarly, the expression of gene, product(s) in other hosts such as of interest in yeast, mammalian cell lines, bacterial and plant systems, can therefore be placed under the regulation of not one, but under the control of multiple (at least two) and different gene promoters, thereby providing a means for dramatically increasing production of proteins of interest in a given host.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A multiple promoter platform for expression of a protein or gene product in a microorganism comprising two or more expression vectors having a nucleic acid molecule encoding a protein to be expressed, wherein each vector has a different promoter operably linked to the nucleic acid molecule.

2. The multiple promoter platform according to claim 1 having three expression vectors.

3. The multiple promoter platform according to claim 1 or 2 wherein the expression vectors are circular plasmids or linear DNA expression cassettes.

4. The multiple promoter platform according to any one of claims 1 to 3 wherein the promoter is in operative association with one or more of secretion signals, pro- and/or core-regions of endogenous proteins, or selection markers.

5. The multiple promoter platform according to any one of claims 1 to 4 wherein the promoter is derived or obtained from the group consisting of hex1, cbh1, cbh2, xyn1, xyn2, xyn3, egl1, egl2, egl3, egl4 and egl5 and apr1.

6. The multiple promoter platform according to any one of claims 1 to 5 wherein the expression vectors contain an secretion signal sequence.

7. The multiple promoter platform according to claim 6 wherein the secretion signal sequence is selected from the group consisting of CBH1, CBH2, XYN1, XYN2, EGL1, EGL2, EGL3, EGL4, and EGL5.

8. The multiple promoter platform according to any one of claims 1 to 7 wherein the vectors contain a selection marker.

9. The multiple promoter platform according to claim 8 wherein the selection marker is selected from the group consisting of amdS, phleo, hphB, als, agrB, trp1, and pyr4.

10. The multiple promoter platform according to claim 1 or 2 wherein the expression vectors are selected from the group consisting of pHEX1, pCBH1corlin, pCBH2sigpro, pCBH2cbmlin, pXYN2sigpro, pEG2sigpro, pEG2cbmlin, and pHEN54RQ.

11. The multiple promoter platform according to any one of claims 1 to 10 wherein the nucleic acid molecule encodes a protein selected from the group consisting of enzymes, cell signalling and ligand binding proteins, structural proteins, proteins or peptides of therapeutic/pharmaceutical use, antibodies, human growth factor, and tissue plasminogen activator.

12. The multiple promoter platform according to claim 11 wherein the protein is phytases, cellulases, xylanases, beta-glucanases, amylases, lipases, mannanases, mannosidases, galactosidases, arabinosidases, xylosidases, glucosidases, glucuronidases, acetyl esterases and proteases.

13. A host cell containing a multiple promoter platform according to any one of claims 1 to 12.

14. A method of producing a protein comprising:
providing a host cell according to claim 13; and
culturing of the host cell such that the protein of interest is expressed by the cell.

15. The method according to claim 14 further comprising obtaining or purifying the expressed protein.

16. The method according to claim 14 or 15 wherein the host cell is transformed with three different expression vectors.

17. The method according to any one of claims 14 to 16 wherein the host cell is a filamentous fungus, yeast, mammalian, plant or bacterial cell.

18. The method according to claim 17 wherein the host cell is a filamentous fungus.

19. The method according to claim 18 wherein the filamentous fungus is Trichoderma reesei.
